(19) United States
(12) Patent Application Publication
(10) Pub. No.: US 2008/0196713 A1
(43) Pub. Date: Aug. 21, 2008

(54) Inhalation Devices

(75) Inventors: Allen John Pearson, Cambridgeshire (GB); Paul Kenneth Rand, Hertfordshire (GB)

Correspondence Address: GLAXOSMITHKLINE;CORPORATE INTELLECTUAL PROPERTY, MAI B482, FIVE MOORE DR., PO BOX 13398, RESEARCH TRIANGLE PARK, NC, 27709-3398, US

(73) Assignee: Glaxo Group Limited

(21) Appl. No.: 11/908,510

(22) PCT Filed: Mar. 16, 2006
(86) PCT No.: PCT/GB06/00966

§ 371 Date: Sep. 13, 2007

(51) Int. Cl. A61M 15/00

(52) U.S. Cl. 128/200.23

(57) ABSTRACT

An inhaler for delivering medicament by inhalation comprises a canister which comprises a body which includes a base and a head and defines a chamber containing medicament, and a valve stem which extends from the head of the body and from which medicament is in use delivered on actuation of the canister. The inhaler further comprises an actuator comprising a main body comprising a housing receiving the canister, and an actuation mechanism for actuating the canister. The actuation mechanism comprises a loading member which is fitted to the head of the canister body and includes a loading section which is located at a distance spaced from the base of the body of the canister and, in use, acted upon to drive the loading member in an actuating direction from a first, rest position to a second, actuated position in which the canister is actuated to deliver medicament. The actuation mechanism further comprises at least one actuating member which is actuatable by a user to drive the loading member in the actuating direction to the actuated position, such as to actuate the canister to deliver medicament. The at least one actuating member is pivotally coupled to the housing for pivotal movement relative to the housing from a first, rest position to a second, actuated position in which the loading member is driven in the actuating direction to the actuated position, such as to actuate the canister to deliver medicament. The at least one actuating member comprises a gripping element which extends along a length of the housing and is configured to be gripped and depressed by the user in actuating the canister.

### FIELD OF THE INVENTION

The present invention relates to an actuator for an inhaler for administering medicament by inhalation and to an inhaler including the same. The invention is particularly, but not exclusively, concerned with an actuator for a pressurised metered dose inhaler (pMDI).

### BACKGROUND OF THE INVENTION

pMDIs are well known in the art of inhalation devices. It is therefore not necessary to describe the construction and operation of a pMDI other than in bare essentials.

A pMDI comprises a canister and an actuator housing. The housing is generally tubular, although this is not essential, and generally formed of a plastics material, for instance by moulding. The canister comprises an open-ended canister, typically made from a metal such as aluminium. The open end of the canister is sealingly capped by a metering valve assembly. The valve assembly typically includes a hollow dispensing member or valve stem which projects from the outlet or business end of the canister. The dispensing member is mounted for sliding movement relative to the canister between an extended position, to which the dispensing member is biased by a biasing mechanism in the valve assembly, typically a return spring, and a depressed position.

In use, the sealed canister contains a pressurised medicinal aerosol formulation. The formulation comprises the medicament and a fluid propellant, and optionally one or more excipients and/or adjuvants. The medicament is typically in solution or suspension in the formulation. The propellant is typically a CFC-free propellant, suitably a liquid propellant, and may for example be HFA-134a or HFA-227.

Movement of the dispensing member from the extended position to the depressed position results in a metered dose of the aerosol formulation being dispensed from the canister through the dispensing member. Typically, the metering valve assembly is provided with a metering chamber of defined volume. In the extended position of the dispensing member, the content of the canister is placed in fluid communication with the metering chamber through the dispensing member so that the metering chamber is filled with the aerosol formulation. When the dispensing member is depressed, the metering chamber is isolated from the canister inner volume and placed in fluid communication with the external environment through the dispensing member. Thus, the defined volume of the aerosol formulation in the metering chamber is discharged to the external environment via the dispensing member.

Such metering valve assemblies are well known in the art and can be obtained from inter alia Bespak Plc (King's Lynn, Norfolk, United Kingdom) and Valois S. A. S. (Le Neubourg, France).

The housing typically comprises an internal passageway having an open end. The canister is slidable into the internal passageway through the open end with the canister being inserted valve assembly first into the internal passageway. A stem block, which receives the dispensing member of the canister when the canister is received in the housing in a “rest position”, has a passageway with an inlet end for receiving the dispensing member and an outlet end, which faces a dispensing outlet of the housing, typically a mouthpiece or a nasal nozzle. The stem block holds the dispensing member stationary whereby depression of the canister to its rest position further into the housing to an “actuated position” causes the dispensing member to be displaced from the extended position to the depressed position relative to the canister. A metered dose of the aerosol formulation will thereby be dispensed out of the dispensing outlet of the housing via the internal passageway of the stem block.

In use, a patient in need of a metered dose of the medicinal aerosol formulation concurrently inhales on the dispensing outlet and depresses the canister from the rest position to the actuated position. The inspiratory airflow produced by the patient entrains the metered dose of the medicinal aerosol formulation into the patient's respiratory tract. So, a pMDI of the type described above is a breath-coordinated inhaler.

Inhalers are commonly provided with a dust cap that covers the dispensing outlet when the inhaler is not in use. The dust cap, when applied, prevents foreign material from entering the housing. This prevents the user from inhaling dust or lint, for example, that might otherwise accumulate in the housing. This is of particular importance where the user suffers from asthma or other respiratory conditions, in which the inhalation of foreign material may cause severe irritation.

Developments to pMDIs have included the provision of actuation indicators or dose counters therefor. Such a dose counter is described in PCT Patent Application Nos. WO-A-9856444 (U.S. Pat. No. 6,431,168) and WO-A-2004/001664 (U.S. Ser. No. 10/518,421) to Glaxo Group Limited, the entire contents of each of these patent applications and patents hereby being incorporated herein by reference. The dose counter is fixably secured on the valve assembly end of the canister and includes a display which denotes the number of metered doses of the medicament formulation dispensed from, or remaining in, the canister. The dose counter is preferably permanently secured on the valve assembly end as described in US-A-2003/0136800 or WO-A-2004/065224 (U.S. Ser. No. 10/543,049), the entire contents of each of these patent applications hereby being incorporated herein by reference. The display of the dose counter is visible to the patient through a window provided in the housing. The display may be presented by a plurality of indicator wheels rotatably mounted on a common axle, each wheel having numerals displayed in series around the circumference.

Many actuators have been developed with a view to facilitating the delivery of medicament, examples of which are disclosed in U.S. Pat. No. 3,272,391, U.S. Pat. No. 3,272,392, U.S. Pat. No. 4,678,106, U.S. Pat. No. 5,899,365, U.S. Pat. No. 6,237,812 and WO-A-99/49917.

It is an aim of the present invention to provide an improved actuator for an inhaler for administering medicament by inhalation and an inhaler including the same.

### SUMMARY OF THE INVENTION

In one aspect the present invention provides an inhaler for delivering medicament by inhalation, comprising:

a canister which comprises a body which includes a base and a head and defines a chamber containing medicament, and a valve stem which extends from the head of the body and from which medicament is in use delivered on actuation of the canister; and

an actuator comprising a main body comprising a housing receiving the canister, and an actuation mechanism for actuating the canister;

the actuation mechanism comprising a loading member which is fitted to the head of the canister body and includes a loading section which is located at a distance spaced from the base of the body of the canister and, in use, acted upon to drive the loading member in an actuating direction from a first, rest position to a second, actuated position in which the canister is actuated to deliver medicament, and at least one actuating member which is actuatable by a user to drive the loading member in the actuating direction to the actuated position, such as to actuate the canister to deliver medicament;

the at least one actuating member being pivotally coupled to the housing for pivotal movement relative to the housing from a first, rest position to a second, actuated position in which the loading member is driven in the actuating direction to the actuated position, such as to actuate the canister to deliver medicament; and

the at least one actuating member comprising a gripping element which extends along a length of the housing and is configured to be gripped and depressed by the user in actuating the canister.

The loading section may be in the form of at least one flange or shelf on an outer surface of the loading member.

The housing may include at least one lateral opening in which the at least one actuating member is disposed for depression by the user.

The actuation mechanism may comprise first and second actuating members which are disposed in oppositely-directed relation.

The main body may include a nozzle block which receives the valve stem of the canister.

The housing may include an outlet member through which the user in use inhales.

The outlet member may be a mouthpiece.

In another aspect of the present invention there is provided the actuator of the inhaler of the invention.

Other aspects and features of the invention are set forth in the appended claims and the exemplary embodiments which will now be described with reference to the accompanying Figures of drawings.

### BRIEF DESCRIPTION OF THE DRAWINGS

FIG. 1 illustrates a perspective view of an inhaler in accordance with a first embodiment of the present invention;

FIGS. 2(a) and 2(b) are part-sectional views of an inhaler corresponding closely to FIG. 1, where illustrated respectively in the rest or inoperative configuration and in the actuated configuration;

FIG. 3 illustrates an aerosol canister assembled with a loading member in the inhaler of FIGS. 1 and 2;

FIG. 4 illustrates a part-sectional view of an inhaler in accordance with a second embodiment of the invention in which a dose counter is permanently fixed to an aerosol canister, where illustrated in the rest or inoperative configuration;

FIG. 5 illustrates a part-sectional view of the inhaler of FIG. 4, where illustrated in the actuated configuration;

FIGS. 6(a) to 6(c) are schematic illustrations of the process by which the aerosol canister and the dose counter in the inhaler of FIGS. 4 and 5 are permanently fixed together; and

FIGS. 7(a) to 7(c) correspond to FIGS. 6(a) to 6(c), respectively, but with the dose counter having a different configuration.

### DETAILED DESCRIPTION OF PREFERRED EMBODIMENTS

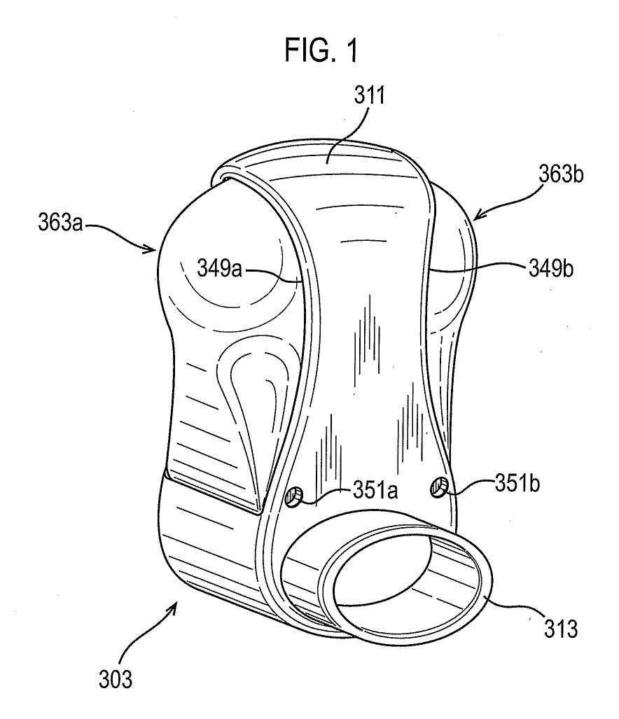

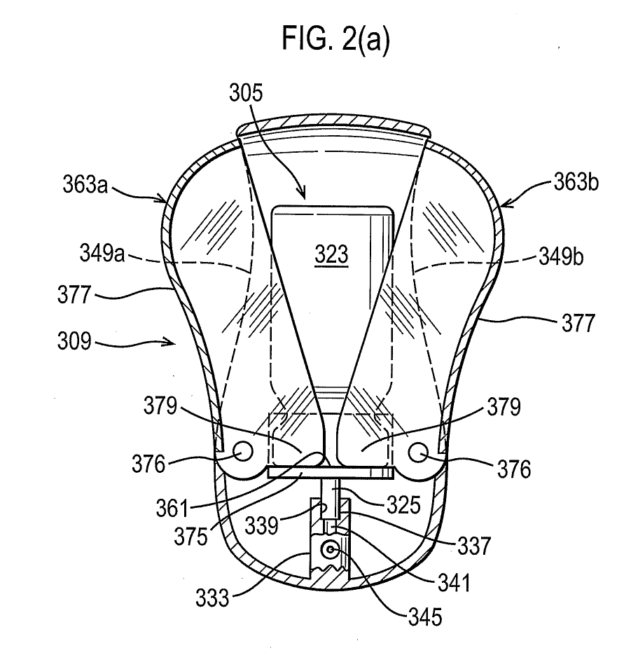

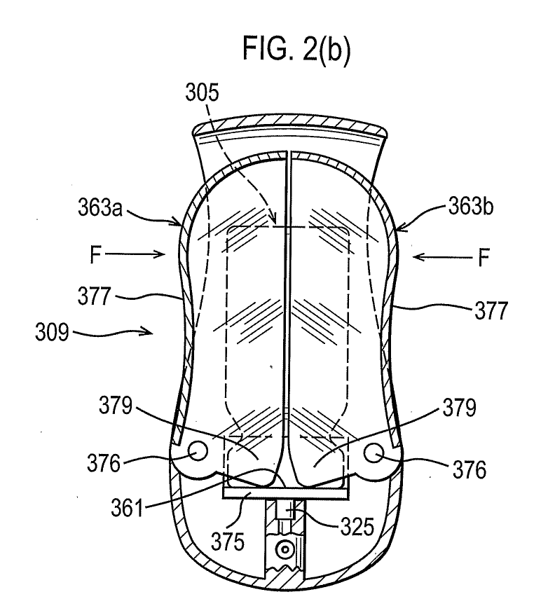

FIGS. 1 to 3 illustrate a hand-held, hand-operable inhaler of the pMDI type in accordance with a first embodiment of the present invention, noting that there are some styling differences between the representations in FIGS. 1 and 2.

The inhaler comprises an actuator which comprises a main body 303, an aerosol canister 305 which is fitted in the main body 303 and contains medicament to be delivered on actuation of the inhaler, and an actuation mechanism 309 which is operable by a user to actuate the inhaler.

The main body 303 comprises a housing 311 in which the canister 305 is in use fitted, and a mouthpiece 313, in this embodiment a tubular element, which is in fluid communication with one, the lower, end of the housing 311 and in use is gripped in the lips of the user. The mouthpiece 313 could instead be configured as a nasal nozzle.

The canister 305 in this embodiment is of standard type, as outlined supra, and comprises a body 323 which includes a base and a head and defines a chamber containing a medicament in a CFC-free propellant under pressure, for example an HFA propellant, a valve stem 325 which extends from the head of the body 323 and an internal metering valve (not illustrated) which is normally biased by an internal valve spring (not illustrated) to a closed position and opened to deliver a metered dose of medicament from the canister 305 when the valve stem 325 is depressed into the canister body 323.

The housing 311 includes a nozzle block 333, in this embodiment disposed to a base surface of the housing 311, for receiving the valve stem 325 of the canister 305.

Referring to FIG. 2, the nozzle block 333 includes a tubular bore 337 for receiving the valve stem 325 of the canister 305, which in this embodiment is co-axial with the longitudinal axis of the housing 311. The tubular bore 337 is open at one, the upper, end thereof and includes an upper section 339 which has an internal dimension which is substantially the same as the outer dimension of the valve stem 325 and a lower section 341 which has a smaller dimension, which sections 339, 341 together define an annular seat for the distal end of the valve stem 325. The tubular bore 337 further includes a laterally-directed spray orifice 345 in the lower section 341 thereof which is configured to direct a spray into and through the mouthpiece 313.

The housing 311 further includes first and second lateral apertures 349a, b, in this embodiment elongate apertures, which are disposed in opposed relation to lateral sides of the mouthpiece 313 and receive actuating members 363a, b of the actuation mechanism 309, as will be described in more detail hereinbelow. The actuating members 363a, b are configured and arranged in the apertures 349a, b such that a gap (not shown) is formed therebetween. The gap functions as an air inlet to the housing 311 in the sense that, when a patient inhales at the mouthpiece 313, air is drawn into the housing 311 through the gap and flows out of the mouthpiece 313 into the patient's respiratory tract. When such inhalation is coordinated with operation of the actuation mechanism 309 to release medicament from the canister 305, the medicament is entrained in this inhalation airflow for delivery to the patient's lungs (or nasal cavity if the mouthpiece 313 is configured as a nasal nozzle).

The housing 311 further includes first and second pivot elements 351a, b which are disposed at the respective lower ends of the lateral apertures 349a, b to which a respective one of the actuating members 363a, b is pivoted, as will be described in more detail hereinbelow. In this embodiment the pivot elements 351a, b each comprise a pair of pivot apertures.

In this embodiment the housing 311 is formed, here by moulding, as a single, integral unit.

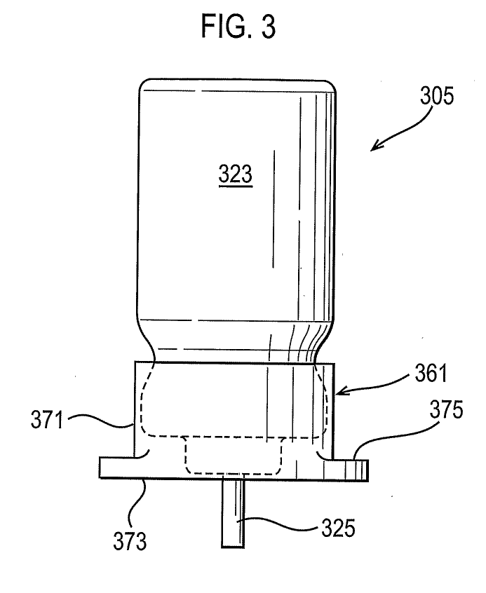

The actuation mechanism 309 comprises a loading member 361 which, as shown clearly in FIG. 3, is fitted over the head of the body 323 of the canister 305, and the first and second actuating members 363a, b which are disposed at the respective ones of the lateral apertures 349a, b in the housing 311 and pivotally mounted to the housing 311 for pivotal movement between a first, rest or inoperative configuration, as illustrated in FIG. 2(a), and a second, actuated configuration, as illustrated in FIG. 2(b), such as to provide for actuation of the canister 305 by engagement with the loading member 361. In this embodiment the loading member 361 is configured as a cap member. Preferably, the loading member 361 is permanently fitted to the head of the body 323 of the canister 305, for instance through use of a split-ring collar as described in US-A-2003/0136800 or WO-A-2004/065224 (U.S. Ser. No. 10/543,049) supra. However, non-permanent fits could be used, for instance a snap fit connection.

In this embodiment the loading member 361 is slideably disposed relative to the nozzle block 333 between a first, rest or inoperative position, as illustrated in FIG. 2(a), and a second, actuated position in which the canister 305 is actuated, as illustrated in FIG. 2(b), and comprises a sleeve 371, here a tubular sleeve, which is a close fit with the outer peripheral wall of the head of the canister 305, an end section 373 at one, the lower, end of the sleeve 371, here which spans the sleeve 371, which engages the head of the body 323 of the canister 305, and which presents an annular loading section 375 which is engaged by the actuating members 363a, b to load the canister 305, as will be described in more detail hereinbelow. The loading section 375 need not necessarily be annular, but instead provide first and second lateral flanges for the actuating members 363a, b to act on.

In this embodiment the actuating members 363a, b each include a pivot element 376 which engages the counterpart pivot element 351a, b in the housing 311, and further each comprise a first, gripping arm 377 which extends across the respective lateral aperture 349a, b in the housing 311 and is configured to be gripped and depressed by the user in actuating the inhaler (e.g. with opposing digits of a user's hand), and a pair of second, loading arms 379 (only one shown per actuating member 363a, b) which extend inwardly from the respective pivot element 376, in a direction substantially orthogonal to the gripping arm 377 so as to straddle the loading member 361, thus defining substantially an inwardly-directed L shape, and is operative to engage the loading section 375 of the loading member 361.

Operation of the actuator will now be described hereinbelow.

The user first takes the actuator in the rest or inoperative configuration, as illustrated in FIG. 2(a), in one hand.

The user then takes the mouthpiece 313 in his/her lips, and, in co-ordination with an inhalation breath, actuates the inhaler by depressing the gripping arms 377 of the actuating members 363a, b with one or more digits of the hand holding the actuator.

As shown in FIG. 2(b), depression of the gripping arms 377 of the actuating members 363a, b causes the inward rotation of the actuating members 363a, b, such that the loading arms 379 of the actuating members 363a, b drive the loading section 375 of the loading member 361, and hence the loading member 361, downwardly, which downward movement of the loading member 361 drives or pulls the canister body 323 of the canister 305 downwardly in relation to the valve stem 325 of the canister 305 which is held stationary by the nozzle block 333.

This downward movement of the body 323 of the canister 305 in relation to the stationary valve stem 325 actuates the canister 305 to deliver a spray of the medicament dispensed from the valve stem 325 into and through the mouthpiece 313.

On releasing the actuating members 363a, b, the inhaler is returned by the valve return spring to the rest configuration illustrated in FIG. 2(a), ready for subsequent actuation.

Following actuation, the inhaler is removed from the mouth, ready for subsequent actuation.

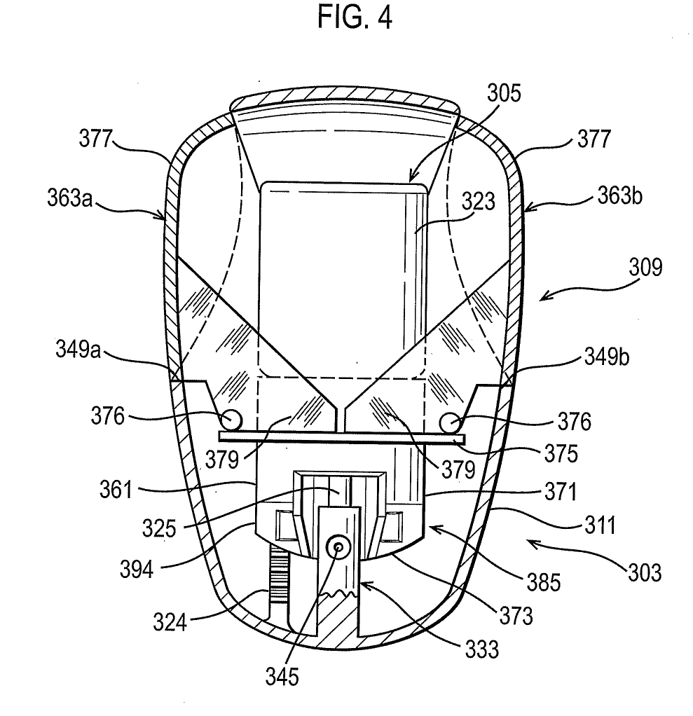

FIGS. 4 to 6 illustrate a hand-held, hand-operable inhaler of the pMDI type in accordance with a second embodiment of the present invention.

The inhaler of this embodiment is very similar to the inhaler of the above-described first embodiment, and thus, in order to avoid unnecessary duplication of description, only the differences will be described in detail, with like parts being designated by like reference signs.

The inhaler of this embodiment differs from that of the first embodiment in the construction of the loading member 361. In this embodiment, the loading member 361 is provided as a modification of the dose counter 385 described in WO-A-2004/001664 (U.S. Ser. No. 10/518,421) supra, which patent applications are incorporated herein by reference.

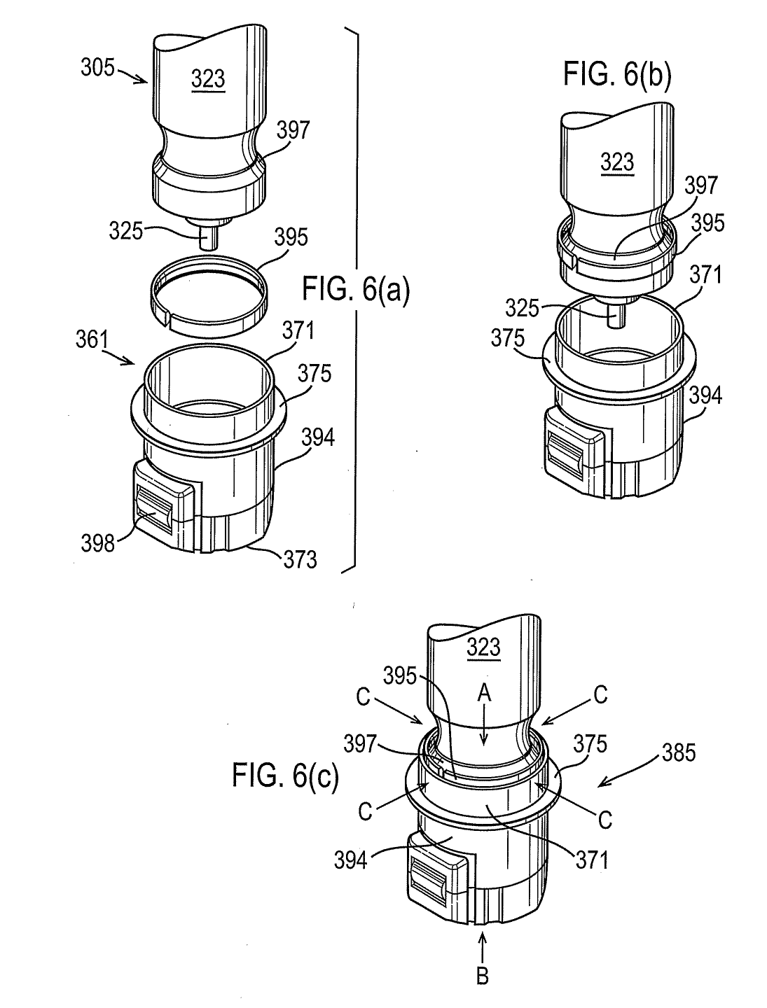

The dose counter 385 is permanently fixed to the head of the canister body 323, to form a unit therewith, through use of a split ring collar 395 detailed in US-A-2003/0136800 or WO-A-2004/065224 (U.S. Ser. No. 10/543,049), all of which are incorporated herein by reference, and as will now be briefly described with reference to FIGS. 6(a)-(c).

FIGS. 6(a)-(c) illustrate the process whereby the collar 395 is fitted around the neck 397 of the canister body 323 and welded to the tubular sleeve 371 of the dose counter housing 394. FIG. 6(a) is an exploded diagram showing the collar 395 being positioned between the canister body 323 and the dose counter housing 394, which is here shown with a counter window 398 in which is a display (not shown) of the number of metered doses of the medicament which are left in the canister 305, or which have been dispensed from the canister 305. FIG. 6(b) shows the collar 395 having been slipped around the neck 397 by opening the collar 395, sliding it over the head of the canister body 323 and then allowing the return force in the collar 395 to close it onto the neck 397. As shown in FIG. 6(c), the collar 395 is slid over the canister 305 in the direction of arrow A thereby causing the collar 395 to radially expand due to the interaction of the inner circumferential surface of the collar 395 with the flaring surface of the neck 397. Meanwhile, the dose counter housing 394 is positioned over the head of the canister body 323 by being moved in the direction of arrow B. In this way, an inner end wall (not shown) of the housing 394 abuts the head of the canister body 323 and the collar 395 is wedged between the inner surface of the sleeve 371 and the neck 397. The collar 395 is then joined to the inner surface of the sleeve 371 by ultrasonic welding at the points indicated by arrows C, thereby permanently securing the dose counter 385 to the canister body 323.

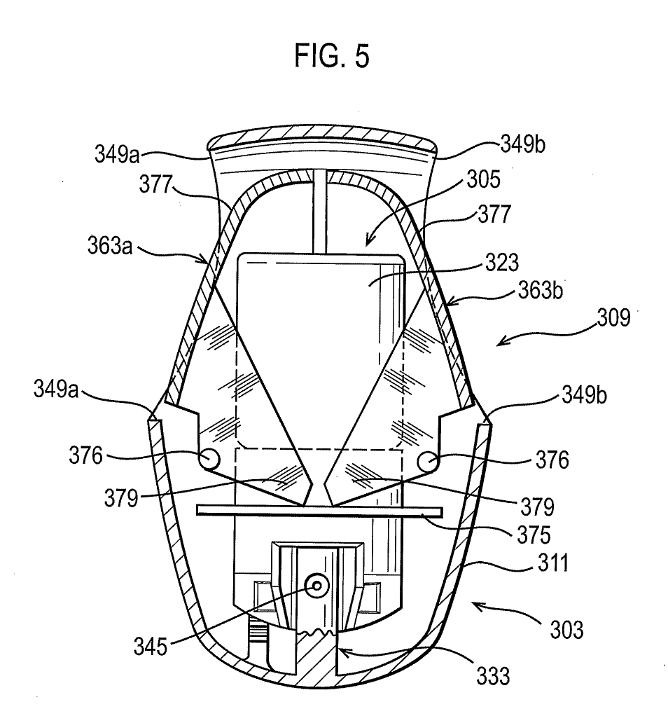

As shown in FIGS. 4 and 5, operation of the inhaler of this second embodiment is the same as for the above-described first embodiment, with the loading section 375 on which the loading arms 379 act being provided as an annular flange or projection on the dose counter housing 394.

FIGS. 4 and 5 show a rack 324 which is disposed to the base surface of the housing 311, beside the nozzle block 333, and which drives the dose counter 385 when the canister 305 is actuated by the actuating mechanism 309, as described in WO-A-2004/001664 (U.S. Ser. No. 10/518,421) supra, incorporated herein by reference.

The display in the dose counter window 398 is updated upon each actuation event and the display is visible to the patient or user through a window or aperture provided in the actuator housing 311 (not shown) at a position to register with the display when the canister-counter unit 305, 385 is mounted in the actuator housing 311.

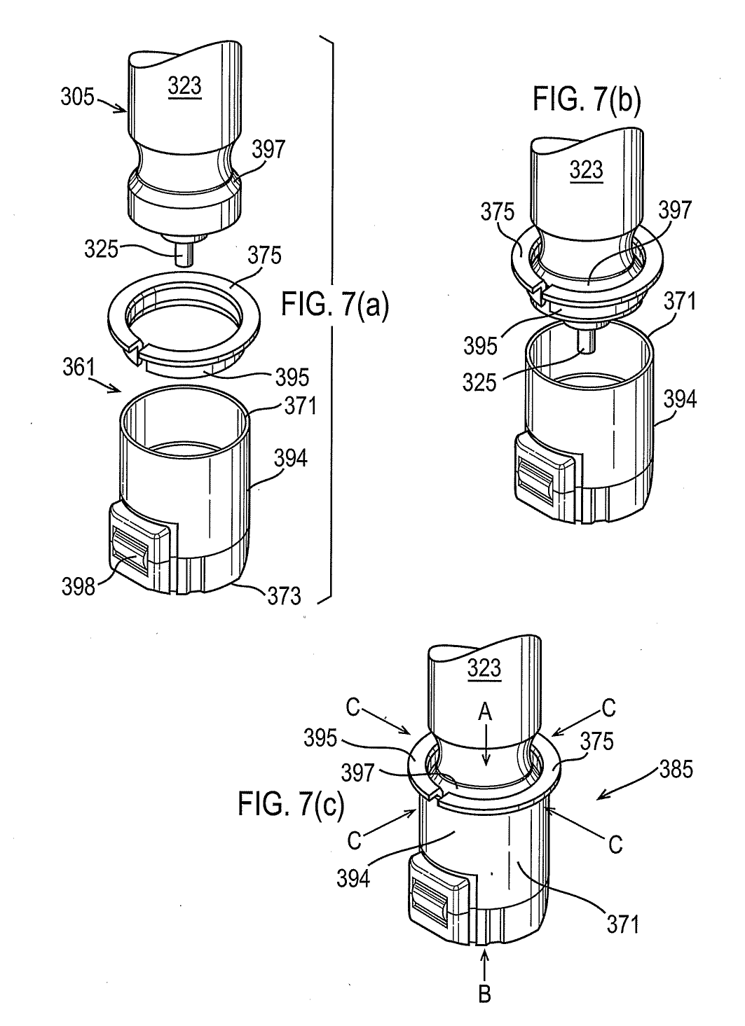

FIGS. 7(a)-(c) show a modification to the second embodiment where the loading section 375 is provided by the split-ring collar 395 instead of the dose counter housing 394. Operation of the inhaler is otherwise the same as described with reference to FIGS. 4 to 6.

As will be appreciated, the loading member 361 may take the form of other accessories which are fixedly connected to the head end of the canister body 323

It will be appreciated that the actuating mechanisms in the illustrated embodiments of the invention provide a mechanical advantage. That is to say, the manual force required to be applied by the user to operate the inhaler (by overcoming the return force of the valve return spring) is less than would otherwise be the case, such as in operation of a standard pMDI where the user has to push down on the base of the canister against the return force of the valve return spring.

Preferably, all of the parts of the actuator of the exemplary embodiments are made from a plastics material, for example by a moulding process.

The medicament contained in the aerosol canister may for the treatment of mild, moderate or severe acute or chronic symptoms or for prophylactic treatment. The medicament is suitably for treating respiratory diseases, e.g. asthma, chronic obstructive pulmonary disease (COPD), although may be for other therapeutic indications, e.g. treating rhinitis.

Appropriate therapeutic agents or medicaments may thus be selected from, for example, analgesics, e.g., codeine, dihydromorphine, ergotamine, fentanyl or morphine; anginal preparations, e.g., diltiazem; antiallergics, e.g., cromoglycate (e.g. as the sodium salt), ketotifen or nedocromil (e.g. as the sodium salt); antiinfectives e.g., cephalosporins, penicillins, streptomycin, sulphonamides, tetracyclines and pentamidine; antihistamines, e.g., methapyrilene; anti-inflammatories, e.g., beclomethasone (e.g. as the dipropionate ester), fluticasone (e.g. as the propionate ester), flunisolide, budesonide, rofleponide, mometasone (e.g. as the furoate ester), ciclesonide, triamcinolone (e.g. as the acetonide), 6α,9α-difluoro-11β-hydroxy-16α-methyl-3-oxo-17α-propionyloxy-androsta-1,4-diene-17β-carbothioic acid S-(2-oxo-tetrahydro-furan-3-yl) ester or 6α,9α-Difluoro-17α-[(2-furanylcarbonyl)oxy]-11β-hydroxy-16α-methyl-3-oxo-androsta-1,4-diene-17β-carbothioic acid S-fluoromethyl ester; antitussives, e.g., noscapine; bronchodilators, e.g., albuterol (e.g. as free base or sulphate), salmeterol (e.g. as xinafoate), ephedrine, adrenaline, fenoterol (e.g. as hydrobromide), formoterol (e.g. as fumarate), isoprenaline, metaproterenol, phenylephrine, phenylpropanolamine, pirbuterol (e.g. as acetate), reproterol (e.g. as hydrochloride), rimiterol, terbutaline (e.g. as sulphate), isoetharine, tulobuterol or 4-hydroxy-7-[2-[[2-[[3-(2-phenylethoxy)propyl]sulfonyl]ethyl]amino]ethyl-2(3H)benzo-thiazolone; PDE4 inhibitors e.g. cilomilast or roflumilast; leukotriene antagonists e.g. montelukast, praniukast and zafirlukast; [adenosine 2a agonists, e.g. 2R,3R,4S,5R)-2-[6-Amino-2-(1S-hydroxymethyl-2-phenyl-ethylamino)-purin-9-yl]-5-(2-ethyl-2H-tetrazol-5-yl)-tetrahydro-furan-3,4-diol (e.g. as maleate)]; [α4 integrin inhibitors e.g. (2S)-3-[4-({[4-(aminocarbonyl)-1-piperidinyl]carbonyl}oxy)phenyl]-2-[((2S)-4-methyl-2-{[2-(2-ethylphenoxy)acetyl]aminoypentanoyl)amino]propanoic acid (e.g as free acid or potassium salt)], diuretics, e.g., amiloride; anticholinergics, e.g., ipratropium (e.g. as bromide), tiotropium, atropine or oxitropium; hormones, e.g., cortisone, hydrocortisone or prednisolone; xanthines, e.g., aminophylline, choline theophyllinate, lysine theophyllinate or theophylline; therapeutic proteins and peptides, e.g., insulin or glucagons. It will be clear to a person skilled in the art that, where appropriate, the medicaments may be used in the form of salts, (e.g., as alkali metal or amine salts or as acid addition salts) or as esters (e.g., lower alkyl esters) or as solvates (e.g., hydrates) to optimise the activity and/or stability of the medicament and/or to minimise the solubility of the medicament in the propellant.

Preferably, the medicament is an anti-inflammatory compound for the treatment of inflammatory disorders or diseases such as asthma and rhinitis.

Preferably, the medicament is formulated in a hydrofluoroalkane propellant, such as HFA-134a or HFA-227 or a combination thereof.

Preferably, the medicament is an anti-inflammatory steroid, such as a corticosteroid, for instance fluticasone, e.g. as the propionate ester, or a long acting beta agonist (LABA), such as salmeterol, e.g. as the xinafoate salt, or a combination thereof.

Preferred medicaments are salmeterol, salbutamol, albuterol, fluticasone and beclomethasone and salts, esters or solvates thereof, for instance fluticasone propionate, albuterol sulphate, salmeterol xinafoate and beclomethasone diproprionate.

The medicament may also be a glucocorticoid compound, which has anti-inflammatory properties. One suitable glucocorticoid compound has the chemical name: 6α,9α-Difluoro-17α-(1-oxopropoxy)-11β-hydroxy-16α-methyl-3-oxo-androsta-1,4-diene-17β-carbothioic acid S-fluoromethyl ester (fluticasone propionate). Another suitable glucocorticoid compound has the chemical name: 6α,9α-difluoro-17α-[(2-furanylcarbonyl)oxy]-11β-hydroxy-16α-methyl-3-oxo-androsta-1,4-diene-17β-carbothioic acid S-fluoromethyl ester. A further suitable glucocorticoid compound has the chemical name: 6α,9α-Difluoro-11β-hydroxy-16α-methyl-17α-[(4-methyl-1,3-thiazole-5-carbonyl)oxy]-3-oxo-androsta-1,4-diene-17β-carbothioic acid S-fluoromethyl ester.

Other suitable anti-inflammatory compounds include NSAIDs e.g. PDE4 inhibitors, leukotriene antagonists, iNOS inhibitors, tryptase and elastase inhibitors, beta-2 integrin antagonists and adenosine 2a agonists.

The medicaments may be delivered in combinations. As an example, there may be provided salbutamol (e.g. as the free base of the sulphate salt) or salmeterol (e.g. as the xinafoate salt) in combination with an anti-inflammatory steroid, such as beclomethasone (e.g. as an ester, preferably dipropionate) or fluticasone (e.g. as an ester, preferably propionate).

Finally, it will be understood that the present invention has been described in its exemplary embodiments and can be modified in many different ways without departing from the scope of the invention as defined by the appended claims.

Also, as regards the provision of reference signs in the appended claims, it is to be understood that reference signs are provided only for illustrative purposes and are not intended to confer any limitation to the claimed invention.

What is claimed is:

1. An inhaler for delivering medicament by inhalation, comprising:
a canister which comprises a body which includes a base and a head and defines a chamber containing medicament, and a valve stem which extends from the head of the body and from which medicament is in use delivered on actuation of the canister; and
an actuator comprising a main body comprising a housing receiving the canister, and an actuation mechanism for actuating the canister;
wherein the actuation mechanism comprises a loading member which is fitted to the head of the canister body and includes a loading section which is located at a distance spaced from the base of the body of the canister and, in use, acted upon to drive the loading member in an actuating direction from a first, rest position to a second, actuated position in which the canister is actuated to deliver medicament, and at least one actuating member which is actuatable by a user to drive the loading member in the actuating direction to the actuated position, such as to actuate the canister to deliver medicament;
wherein the at least one actuating member is pivotally coupled to the housing for pivotal movement relative to the housing from a first, rest position to a second, actuated position in which the loading member is driven in the actuating direction to the actuated position, such as to actuate the canister to deliver medicament; and
wherein the at least one actuating member comprises a gripping element which extends along a length of the housing and is configured to be gripped and depressed by the user in actuating the canister.

2. The inhaler of claim 1, wherein the loading section of the loading member comprises a substantially annular section.

3. The inhaler of claim 1, wherein the at least one actuating member comprises a loading element for engaging the loading section of the loading member when the at least one actuating member pivots from the first, rest position to the second, actuated position to drive the loading member in the actuating direction to the actuated position, such as to actuate the canister to deliver medicament.

4. The inhaler of claim 3, wherein the gripping element extends from a pivot along a length of the housing and the loading element extends inwardly from the pivot.

5. The inhaler of claim 1, wherein the housing includes at least one lateral opening in which the at least one actuating member is disposed for depression by the user.

6. The inhaler of claim 1, wherein the actuation mechanism comprises first and second actuating members which are disposed in oppositely-directed relation.

7. The inhaler of claim 1, wherein the main body includes a nozzle block which receives the valve stem of the canister.

8. The inhaler of claim 3, wherein the housing includes an outlet member through which the user in use inhales.

9. The inhaler of claim 8, wherein the outlet member is a mouthpiece.

10. The inhaler of claim 1, wherein the loading member is permanently fitted to the head of the canister body.

11. The inhaler of claim 1, wherein the loading member is attached to the canister body at a neck thereof.

12. The inhaler of claim 11, wherein the loading member comprises a sleeve having an inner surface in opposed relation to the neck and an annular element which is mounted on the neck and connected to the inner surface.

13. The inhaler of claim 1, wherein the canister body has an outer peripheral surface extending from the base to the head and the loading member has a sleeve which is disposed about the outer peripheral surface adjacent the head and presents the loading section.

14. The inhaler of claim 12, wherein the annular element presents the loading section.

15-17. (canceled)
